(19) United States
(12) Patent Application Publication
(10) Pub. No.: US 2026/0137909 A1
(43) Pub. Date: May 21, 2026

(54) CATHETER ASSEMBLY WITH NEEDLE HOODING ADVANCEMENT FEATURE

(71) Applicant: Becton, Dickinson and Company, Franklin Lakes, NJ (US)

(72) Inventor: Jonathan Karl Burkholz, Salt Lake City, UT (US)

(73) Assignee: Becton, Dickinson and Company, Frankin Lakes, NJ (US)

(21) Appl. No.: 18/949,290

(22) Filed: Nov. 15, 2024

(51) Int. Cl. A61M 25/06

(52) U.S. Cl. CPC A61M 25/0606; A61M 25/065

(57) ABSTRACT

Catheter assemblies vascular access devices using such assemblies are disclosed. The catheter assembly comprises a catheter, an introducer needle, a needle hub and a catheter distal tip support element. The assembly includes an advancement assembly disposed between the needle hub and the distal tip of the introducer needle, the advancement assembly configured to advance the catheter distal tip support element a distance sufficient to advance the distal end of the elongate tubular portion to cover the distal tip of the needle in a hooded position.

### TECHNICAL FIELD

The present disclosure pertains to catheter assemblies including a relatively soft catheter positioned over an introducer needle and a catheter distal tip support that is relatively hard and movable with respect to the catheter that is positioned over the introducer needle.

### BACKGROUND

Intravenous (IV) catheters are commonly used for a variety of infusion therapies. For example, IV catheters is used for infusing normal saline solution, various medicaments, or total parenteral nutrition into a patient. IV catheters may also be used for withdrawing blood from the patient.

A common type of IV catheter is an over-the-needle peripheral IV catheter. As its name implies, over-the-needle peripheral IV catheters comprise catheter tubing mounted tightly over an introducer needle having a sharp distal tip. The introducer needle and the IV catheter are generally inserted at a shallow angle through the skin into vasculature of the patient with a bevel of the introducer needle facing away from the skin of the patient. During insertion, the sharp distal tip of the introducer needle is distal to a distal end of the IV catheter. Thus, insertion of the IV catheter into the vasculature may follow piercing of the vasculature by the introducer needle.

Generally, the introducer needle is constructed of metal, while the catheter tubing is constructed of a softer material, for example, a thermoplastic or elastomeric material. Softer catheter tubing materials, as opposed to harder materials, are more comfortable for the patient, cause less irritation, and improve indwelling. However, if the catheter tubing is too soft, its columnar strength may not be great enough to resist forces required to penetrate the skin and tissue of the patient.

In some instances, when the introducer needle and the IV catheter are inserted into the patient, there is a tendency for the catheter tubing to buckle, peel back, or move proximally away from the distal tip of the introducer needle. This may occur due to the skin or tissue of the patient pushing on a distal tip of the IV catheter during the insertion process. When the catheter tubing peels back or moves proximally away from the distal tip of the introducer needle, the catheter tubing may adopt an enlarged cross-section, which may require greater insertion force, increased dilation at the insertion site, and/or increased pain for the patient.

There is a need to provide catheter assemblies that improve insertion success, reduce placement related complications, and provide a softer, longer dwelling catheter tubing than those provided with traditionally placed short and mid-line peripheral IV catheters.

### SUMMARY

One aspect of the disclosure relates to a catheter assembly configured for insertion into vasculature of a patient. In one embodiment, the catheter assembly comprises a catheter including a catheter lumen and extending from a distal end of a catheter hub having a proximal end. The catheter comprises a catheter stepped surface disposed within the catheter lumen and having a distal surface with a first inner diameter, a proximal surface with a second inner diameter greater than the first inner diameter, and a shoulder disposed between the distal surface and the proximal surface. The catheter further comprises an elongate tubular portion proximate the catheter stepped surface and having the second inner diameter of the proximal surface and a distal end.

The catheter assembly further includes an introducer needle disposed within the catheter lumen, the introducer needle comprising a sidewall defining a proximal end, a distal tip, and a lumen extending through from the proximal end to the distal tip, the proximal end attached to a needle hub. The catheter assembly further includes a catheter distal tip support element movably disposed within the catheter lumen and over the introducer needle, the catheter distal tip support element comprising a proximal end and a support material having a higher Shore durometer than a Shore durometer of the second material of the elongate tubular portion of the catheter, the catheter distal tip support element movable with respect to the catheter and the introducer needle and having a distal surface configured to contact the shoulder of the catheter and to advance the catheter in distal direction and prevent kinking or buckling of the second material of the elongate tubular portion as the catheter is advanced into the vasculature of the patient.

The catheter assembly further includes an advancement assembly disposed between the needle hub and the distal tip of the introducer needle, the advancement assembly configured to advance the catheter distal tip support element a distance sufficient to advance the distal end of the elongate tubular portion to cover the distal tip of the needle in a hooded position.

### BRIEF DESCRIPTION OF THE DRAWINGS

FIG. 1A is a top view of a catheter assembly according to one or more embodiments;

FIG. 1B is a cross-sectional view of an over-the-needle catheter of the catheter assembly of FIG. 1A, according to one or more embodiments;

FIG. 1C is an upper perspective view of the distal end of the over-the-needle catheter of FIG. 1B, according to one or more embodiments;

FIG. 1D is an upper perspective view of a distal end of an example introducer needle of the catheter assembly of FIG. 1A, according to one or more embodiments;

FIG. 2 is a cross-sectional view of a portion of a catheter assembly including an introducer needle, a catheter over the introducer needle and a reinforced distal catheter tip according to one or more embodiments;

FIG. 3A is a cross-sectional view of a catheter assembly according to a first embodiment of the disclosure with the catheter in a pre-insertion position;

FIG. 3B is a cross-sectional of the catheter adapter assembly shown in FIG. 3A with the catheter in a partially advanced position over the distal tip of the introducer needle;

FIG. 4 is a top cross-sectional of a second embodiment of a catheter assembly showing the over-the-needle catheter in a pre-insertion position;

FIG. 5 is an isometric view of the catheter assembly shown in FIG. 4 with the over-the-needle catheter in a pre-insertion position;

FIG. 6 is a cross-sectional view of a portion of the catheter assembly shown in FIG. 5 taken along line 6-6; and

FIG. 7 is a side view of an advancement feature and.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

Embodiments of the present disclosure provide catheter assemblies which can be used with a variety of vascular access devices (VAD) and in various methods and systems. One or more embodiments provide catheter assemblies that provide one or more of improved insertion success, reduced placement related complications, and provide a softer, longer dwelling a softer, longer dwelling catheter tubing than those provided with traditionally placed short and mid-line peripheral IV catheters. Embodiments of the catheter assembly and systems of this disclosure provide for placement of a VAD with a much softer, and longer dwelling, catheter material via an over-the-needle catheter placement device. This is accomplished by providing a movable distal catheter tip support and “hooding” feature and a softer, more compliant and kink resistant catheter material. One or more embodiments also provide a catheter assembly that is configured to place a longer catheter for deeper vein placements that often have a steeper insertion angle and tighter angle changes that can be susceptible to kinking or buckling. According to one or more embodiments, a catheter assembly and system is provided that is less complicated than existing catheter assemblies.

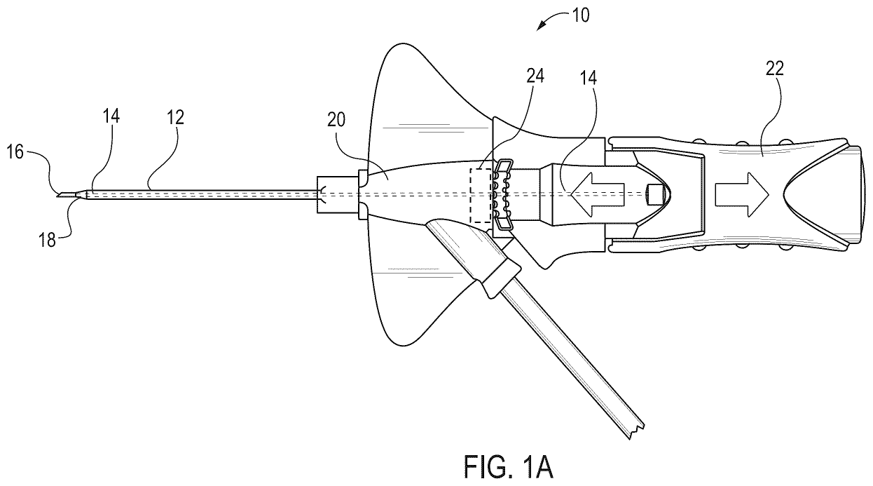

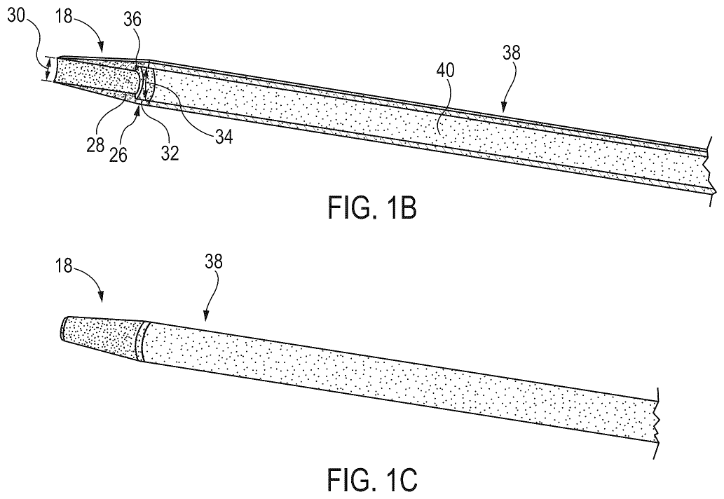

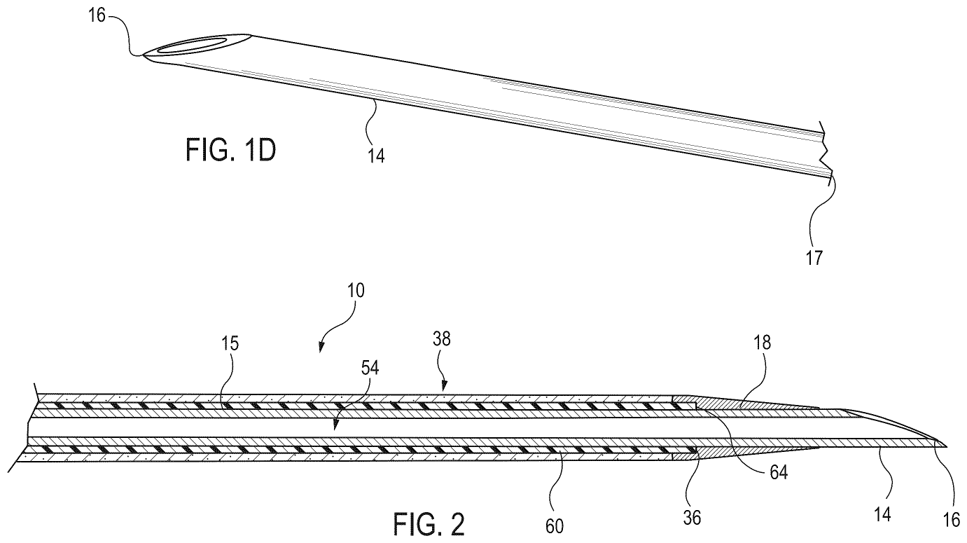

Embodiments of the present disclosure generally relate to a catheter assembly configured for insertion into vasculature of a patient and related devices and methods. Referring now to FIG. 1A, in one or more embodiments, the catheter assembly 10 includes an over-the-needle catheter 12 and an introducer needle 14. FIG. 1A illustrates the introducer needle 14 in an insertion position for insertion into a patient. The introducer needle 14 comprises a distal tip, a proximal end and a notch (or slot) 52. In one or more embodiments, when the introducer needle 14 is in the insertion position, a sharp distal tip 16 of the introducer needle 14 is disposed beyond a distal tip 18 of the over-the-needle catheter 12.

In one or more embodiments, the catheter assembly 10 includes a catheter adapter 20. As shown, a proximal end of the over-the-needle catheter 12 is secured within the catheter adapter 20, and the over-the-needle catheter 12 extends distally from the catheter adapter 20. A proximal end of the introducer needle 14 is secured within a needle hub 22 of the catheter assembly 10.

The catheter assembly 10 shown includes any type of catheter assembly having an over-the-needle catheter and introducer needle. Although the catheter assembly 10 illustrated in FIG. 1A is shown as having an integrated extension tube, it is understood that the catheter assembly 10 is straight or non-integrated. Non-limiting examples of integrated IV catheter systems includes, for example, the BD NEXIVA™ Closed IV Catheter System, the BD NEXIVA™ DIFFUSICS™ Closed IV Catheter System, or the Becton Dickinson PEGASUS™ Safety Closed IV Catheter System. A non-limiting example of a straight or non-integrated catheter assembly is the BD INSYTE™ AUTOGUARD™ Shielded IV Catheter. In one or more embodiments, the catheter assembly 10 includes any variety of needle safety mechanism, which may shield the distal tip 16 of the introducer needle 14.

In one or more embodiments, the catheter assembly 10 includes any suitable type of blood control septum, which is disposed within a lumen of the catheter adapter 20. FIG. 1A illustrates an example blood control septum 24, according to one or more embodiments. In one or more embodiments, the introducer needle 14 is proximally withdrawn through the septum 24 after the over-the-needle catheter 12 is properly placed within the vasculature. In one or more embodiments, the septum 24 is elastomeric. In one or more embodiments, the septum 24 may divide the lumen of the catheter adapter 20 into a proximal chamber and a distal chamber.

Referring now to FIG. 1B, in one or more embodiments, the over-the-needle catheter 12 includes the distal tip 18, which is constructed of the first material having a first Shore durometer value. In one or more embodiments, the first material is rigid or semi-rigid. In one or more embodiments, the over-the-needle catheter 12 includes a first stepped surface 26, which in some embodiments is referred to a catheter stepped surface 26 that is annular. In other embodiments, the stepped surface 26 may comprise one or more discrete protrusions protruding from an interior surface of the catheter lumen 40 of the over-the-needle catheter 12 that provide a stepped surface 26, which forms a shoulder. In one or more embodiments, the first stepped surface 26 includes a rigid or semi-rigid distal surface 28 with a first inner diameter 30, a proximal surface 32 with a second inner diameter 34 greater than the first inner diameter 30, and a rigid or semi-rigid first transition surface defining a shoulder 36 disposed between the distal surface 28 and the proximal surface 32. The first inner diameter 30 is uniform according to one or more embodiments. In some embodiments the second inner diameter 34 is uniform.

In one or more embodiments, an entirety of the distal tip is constructed of the first material, which may also be referred to as the distal tip material. In one or more embodiments, at least the distal surface 28 and the first transition surface defining the shoulder 36 of the first stepped surface 26 is part of the distal tip 18 of the over-the-needle catheter 12 and thus constructed of the first material. In one or more embodiments, the distal surface 28, the first transition surface defining the shoulder 36, and the proximal surface 32 of the first stepped surface 26 is part of the distal tip 18 and thus constructed of the first material. In some embodiments, the distal type includes fenestrations or openings (not shown).

In one or more embodiments, the catheter 12 includes an elongate tubular portion 38 proximate and coupled to the distal tip 18 of the over-the-needle catheter 12. In one or more embodiments, the tubular portion 38 is constructed of a second material. In one or more embodiments, a Shore durometer value of the first material is equal to or higher (a harder material) than a Shore durometer value of the second material. In one or more embodiments, the Shore durometer of the first material and the second material are equal, and in some embodiments, the first material and the second material comprise, consist of or consist essentially of the same material. In one or more embodiments, the distal tip 18 and the tubular portion 38 includes a catheter lumen 40 extending there through. In one or more embodiments, the first stepped surface 26 is disposed within the catheter lumen 40.

Referring now to FIG. 1C, in one or more embodiments, at least a portion of an outer surface of the distal tip 18 is tapered, which may facilitate entry of the over-the-needle catheter 12 into the skin and tissues of the patient behind the distal tip 16 of the introducer needle 14 as the distal tip 16 cuts a pathway. Referring to FIG. 1D, the catheter assembly 10 includes the introducer needle 14 having a length extending from the distal tip 16.

In one or more embodiments, the first material and/or the second material includes a composite material. In one or more embodiments, the first material includes one or more of the following: silicone, polyurethane, metal, plastic, and another suitable material. In one or more embodiments, the second material may one or more of the following: silicone, polyurethane, metal, plastic, and another suitable material. In one or more embodiments, the first material includes a first polyurethane, and the second material includes a second polyurethane. In one or more embodiments, the first material includes a first silicone, and the second material includes a second silicone. In one or more embodiments, the first material includes metal and/or the second material includes plastic. In one or more embodiments, the second material includes a flexible material. In one more embodiments, the second material comprises a soft polymeric material, for example, silicone rubber, nylon, polyurethane, fluoroethylene polymer (FEP), polytetrafluoroethyene polymer (PTFE), polyethylene terephthalate (PET), latex, and thermoplastic elastomers.

In one or more embodiments, the second material includes a softer material than the first material, which is more comfortable for the patient and result in fewer indwelling problems. In one or more embodiments, the softer second material may allow the over-the-needle catheter 12 to be inserted into the vasculature longer before complications arise.

Referring to FIG. 2, the catheter assembly includes a catheter distal tip support element 60 movably disposed within the catheter lumen 40 of the over-the-needle catheter 12 and over the introducer needle 14. The catheter distal tip support element 60 comprises a support material having a Shore durometer that is equal to or greater than a Shore durometer of the second material which makes up the elongate tubular portion 38 of the over-the-needle catheter 12. In embodiments in which the Shore durometer of the first material is greater than the Shore durometer of the second material, and the first material is harder than the second material.

The catheter distal tip support element 60 is movable in a distal and proximal direction with respect to the over-the-needle catheter 12 and the introducer needle 14. The catheter distal tip support element 60 in some embodiments has an outer diameter 62 and a support distal surface 64 configured to contact the shoulder 36 of the over-the-needle catheter 12 and to advance the over-the-needle catheter 12 in distal direction and prevent kinking or buckling of the second material of the over-the-needle catheter 12 as the over-the-needle catheter 12 is advanced into the vasculature of the patient. In one or more embodiments, the support distal surface 64 may contact the shoulder 36 of the over-the-needle catheter 12 when the introducer needle 14 is in the insertion position, such that proximal movement of the distal tip 16 relative to the introducer needle 14 is inhibited and compressive forces necessary for the distal tip 16 to penetrate the skin are transferred to the distal tip 18 of the over-the-needle catheter 12. In one or more embodiments, the contact between the support distal surface 64 and the shoulder 36 when the introducer needle 14 is in the insertion position may allow the second material to be even softer than a standard catheter body. In one or more embodiments, the second material may allow the tubular portion 38 to bend when the catheter adapter 20 is secured to the skin of the patient, while the first material may facilitate successful insertion of the over-the-needle catheter 12 into the vasculature.

In one or more embodiments, the first transition surface defines a shoulder 36 that is disposed at about 90 degrees with respect to a longitudinal axis of the catheter assembly 10, as illustrated, for example, in FIG. 1B. In one or more embodiments, the first transition surface defining the shoulder 36 is disposed at an angle other than 90 degrees with respect to the longitudinal axis of the catheter assembly 10. In one or more embodiments, the first transition surface defining the shoulder 36 is smooth and/or planar. In one or more embodiments, the first transition surface defining the shoulder 36 is uneven, rough, or irregular. In one or more embodiments, the first transition surface defining the shoulder 36 is curved.

FIG. 2 is a cross-sectional view of a catheter assembly 10 including the introducer needle 14 and the over-the-needle catheter 12 with the distal tip 18 according to one or more embodiments.

The catheter assembly 10 is configured for insertion into vasculature of a patient. The catheter assembly 10 includes an over-the-needle catheter 12 which comprises the catheter stepped surface 26 having a distal surface 28 with a first inner diameter 30, a proximal surface 32 with a second inner diameter 34 greater than the first inner diameter 30, and a shoulder 36 disposed between the distal surface 28 and the proximal surface 32.

The catheter assembly 10 further comprises an elongate tubular portion 38 proximate the catheter stepped surface 26 and having the second inner diameter 34 of the proximal surface 32, wherein the shoulder 36, the proximal surface 32, and the distal surface 28 are constructed of a first material having a Shore durometer that is equal to or greater than a Shore durometer a second material of the elongate tubular portion 38. The over-the-needle catheter 12 has a catheter lumen 40 extending therethrough, and the catheter stepped surface 26 is disposed within the catheter lumen 40.

The catheter assembly 10 further comprises an introducer needle 14 disposed within the catheter lumen 40, the introducer needle 14 comprising a sidewall 15 defining a proximal end 17, a distal tip 16 and a needle lumen 54 extending through the sidewall 15 from the proximal end 17 to the distal tip 16.

The catheter assembly 10 further comprises a catheter distal tip support element 60 movably disposed within the catheter lumen 40 and over the introducer needle 14, the catheter distal tip support element 60 comprising a support material having a Shore durometer that is equal to or greater than a Shore durometer of the second material of elongate tubular portion 38 of the catheter, the catheter distal tip support element 60 element movable with respect to the over-the-needle catheter 12 and the introducer needle 14 and having a support distal surface 64 configured to contact the shoulder 36 of the over-the-needle catheter 12 and to advance the over-the-needle catheter 12 in distal direction and prevent kinking or buckling of the second material of the catheter as the catheter is advanced into the vasculature of the patient.

One method often used during catheter placement is referred to as a “hooding” method. During insertion, upon initial flashback of blood confirmation that the need has punctured the targeted vein, clinicians often pause further needle advancement, and partially advance the catheter far enough for the catheter to cover the needle tip and bevel. This helps prevent the bevel from causing unnecessary vein damage during the remainder of the insertion process. Once the catheter is hooded, the angle is typically reduced or lowered, and the catheter can be fully advanced into the vein. Although hooding is currently used as an insertion method, it is fully based on user skill and technique and is often not effective. According to one or more embodiments, a catheter assembly is provided which establishes a mechanically defined position to allow proper and consistent hooding while providing tip support to be able to successfully advance a catheter with much softer material.

Thus, it will be appreciated that the catheter distal tip support element 60 comprises a sleeve as shown that slides over the introducer needle 14 and is sandwiched between the sidewall 15 of the needle and the tubular portion 38 of the over-the-needle catheter 12. In some embodiments, the sleeve comprises a sleeve material having a Shore durometer that is equal to or greater than (a harder material) than a Shore durometer than the second material of the tubular portion 38 of the catheter. In some embodiments, the Shore durometer of the sleeve is equal to or greater than the Shore durometer of the distal tip 18 of the over-the-needle catheter 12. The sleeve material can comprise plastic or metal.

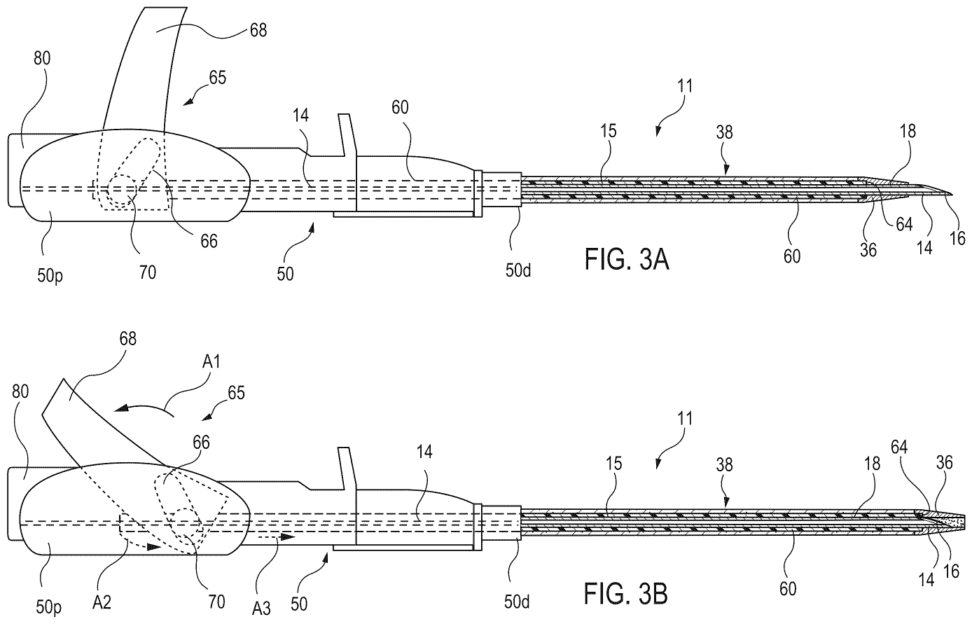

Referring now to FIGS. 3A and 3B, an embodiment of a catheter assembly 11 configured for insertion into vasculature of a patient is shown. The catheter assembly 11 comprises the introducer needle 14 and the catheter 12 as shown and described with respect to FIGS. 1B-1D and FIG. 2. Thus, the catheter assembly 11 comprises the catheter 12 including the catheter lumen 40 and extending from a distal end 50d of a catheter hub 50 having a proximal end 50p.

The catheter 12 of the catheter assembly 11 comprises the catheter stepped surface 26 disposed within the catheter lumen 40 and having the distal surface 28 with a first inner diameter 30, the proximal surface 32 with the second inner diameter 34 greater than the first inner diameter 30, and the shoulder 36 disposed between the distal surface 28 and the proximal surface 32.

The catheter 12 of the catheter assembly 11 further comprises the elongate tubular portion 38 proximate the catheter stepped surface 26 and having the second inner diameter 34 of the proximal surface 32 and the distal tip 18.

The catheter assembly 11 shown in FIGS. 3A and 3B further comprises the introducer needle 14 disposed within the catheter lumen 40, the introducer needle comprising the sidewall 15 defining the proximal end 17, the distal tip 16, and the lumen 54 extending through from the proximal end 17 to the distal tip 16, the proximal end 17 attached to a needle hub 80.

Still referring to FIGS. 3A and 3B, the catheter assembly 11 includes a catheter distal tip support element 60 movably disposed within the catheter lumen 40 and over the introducer needle 14. The catheter distal tip support element 60 comprises a proximal end 60p (shown in FIG. 7) and a support material having a higher Shore durometer than a Shore durometer of the second material of the elongate tubular portion 38 of the catheter 12. The catheter distal tip support element 60 is movable with respect to the catheter 12 and the introducer needle 14 and has a distal surface 64 configured to contact the shoulder 36 of the catheter 12 and to advance the catheter 12 in distal direction and prevent kinking or buckling of the second material of the elongate tubular portion 38 as the catheter 12 is advanced into the vasculature of the patient.

FIGS. 3A and 3b further show the catheter assembly 11 including an advancement assembly 65 disposed between the needle hub 80 and the distal tip 16 of the introducer needle 14. In the embodiment shown, the advancement assembly is shown between the proximal end 50p and the distal end 50d of the catheter hub 50. The advancement assembly 65 is configured to advance the catheter distal tip support element 60 a distance sufficient to advance the distal tip 18 of the catheter 12 to cover the distal tip 16 of the introducer needle 14 in a hooded position.

Referring to FIGS. 3A and 3B, the advancement assembly 65 includes an advancement feature 70 coupled to the catheter distal tip support element 60. In some embodiments, the advancement feature 70 is coupled adjacent to the proximal end 60p of the catheter distal tip support element 60. In some embodiments, the advancement feature 70 extends transverse to the catheter distal tip support element 60. In some embodiments, the advancement feature 70 is the form of a rod or a beam that is affixed to the catheter distal tip support element 60.

Still referring to FIGS. 3A and 3B, the advancement assembly 65 further comprises a lever element 68 engaged with the advancement feature 70. In specific embodiments, the lever element comprises a slot 66, and the advancement feature 70 is engaged with the slot 66. In one or more embodiments of the catheter assembly 11, the lever element 68 and the advancement feature 70 are engaged with the catheter hub 50. According to one or more embodiments, engaged with the catheter hub means that the lever element 68 and the advancement feature 70 are attached, fastened to or assembled to a portion of the catheter hub. For example, the advancement assembly 65, including one or more of the lever element 68 and the advancement feature 70 are contained within a pocket or a hollowed area of the catheter hub 50. In some embodiments, one or more of the lever element 68 and the advancement feature 70 are affixed to the catheter hub 50 by a bracket or any other suitable method of affixing the features to the catheter hub 50. In one or more embodiments, the components are engaged with or affixed to the catheter hub 50 so that the advancement assembly can advance the catheter distal tip 18 a predetermined distance to hood the distal tip 18 of catheter 12 over the distal tip 16 of the introducer needle 14. In some embodiments, the lever element 68 and the advancement feature 70 are engaged with the proximal end 50p of the catheter hub 50.

Thus, with continued reference to FIG. 3 A and 3B, the advancement assembly 65 applies a user applied displacement force on the catheter distal tip support element 60, which applies a force to the distal tip 18 of the catheter 12 to advance the catheter assembly 11 and catheter 12 to a “hooded” position. As discussed herein, “hooding” a catheter refers to the practice of advancing the catheter 12 to cover the introducer needle distal tip 16 after the introducer needle 14 punctures the vein or artery of a patient. This allows the remainder of the catheter placement and advance to be performed more safely with less risk of damage to the vein or artery.

During the initial insertion, the catheter distal tip support element 60 provides support to the catheter distal tip 18 to maintain its position relative to the distal tip 16 of the introducer needle 14, so the softer catheter material does not compress or buckle during penetration through the skin of a patient. The advancement assembly 65 provides simultaneous displacement forces on both the catheter hub 50 and the distal tip 18 of the catheter 12 to “hood” the introducer needle 14 after vein puncture, and during the rest of the insertion process (lowering the angle, advancement, threading, and needle removal).

FIG. 3A shows the lever and the advancement assembly 65 in an initial position with the advancement feature 70 at the lower portion of the slot 66. Moving the lever element 68 in a distal direction, for example, by rotating the lever element 68 in the direction of arrow A1 moves the advancement feature 70 in a distal direction shown by arrows A2 and A3 in FIG. 3B, which causes the catheter 12 to hood the distal tip 16 of the introducer needle 14.

In some embodiments, the lever element 68 is pivotable about the advancement feature 70. In other embodiments, applying a pushing force on the lever element 68 advances the advancement feature. In some embodiments, pivoting the lever element 68 advances the catheter distal tip support element 60 a distance sufficient to advance the distal tip 18 of the catheter 12 to cover the distal tip 16 of the introducer needle 14 in a hooded position. The lever elements 68 of some embodiments is pivotable in a distal direction. In other embodiments, the lever element 68 is pivotable in a proximal direction. In some embodiments, the lever element is displaced by rotation to advance the advancement feature 70.

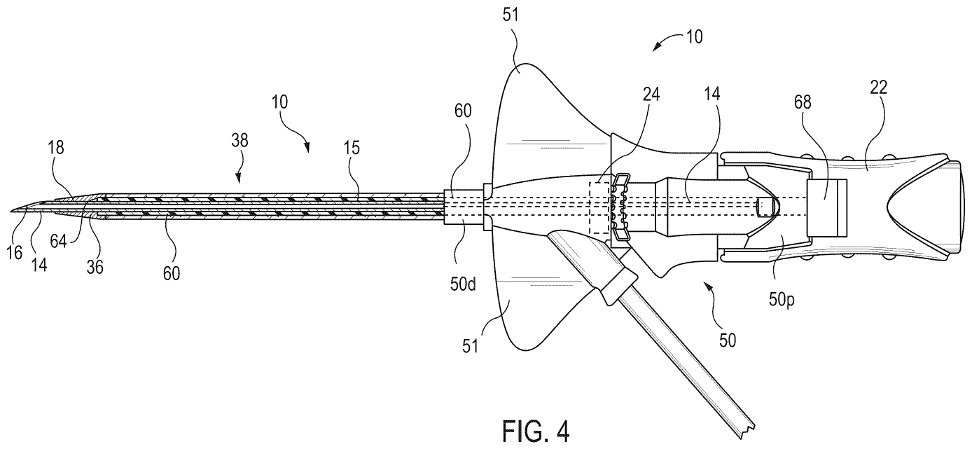

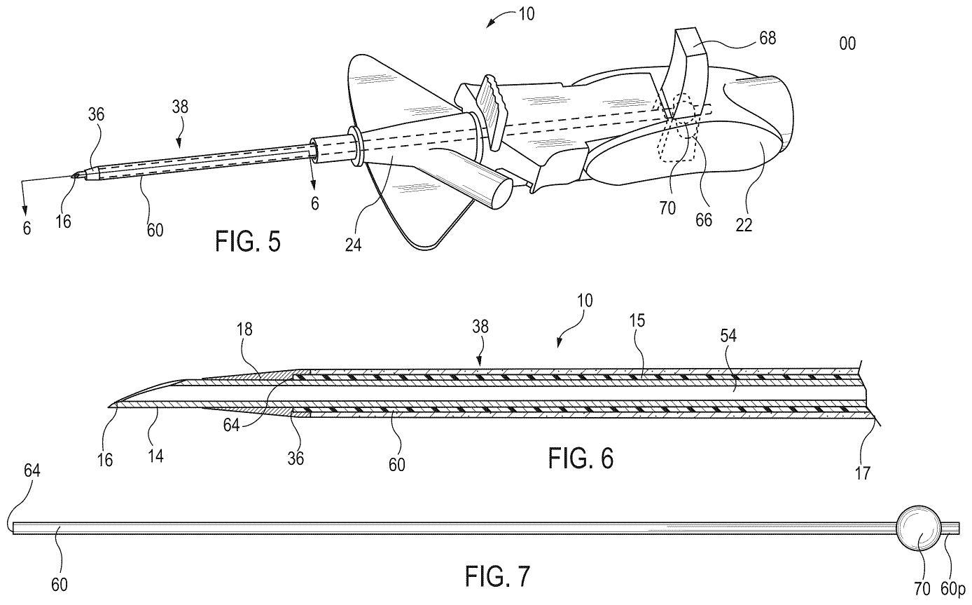

Referring now to FIGS. 4-6, a second embodiment of a catheter assembly 10 is shown including a different type of catheter hub 50 having an advancement assembly 65 including a lever element 68 adjacent to the proximal end 50p of the catheter hub 50. The catheter assembly 10 includes catheter wings 51 adjacent to the distal end 50d of the catheter hub 50. Operation of the catheter assembly 10 shown in FIGS. 4 and 5 is similar to the operation of the catheter assembly 11 shown in FIGS. 3A and 3B.

The catheter assemblies described in this disclosure may be applied to a wide variety of medical devices. They may be applied to peripheral intravenous catheters (PIVC), long PIVC, mini-midline, midline, arterial, and port access vascular access devices. Embodiments of the catheter assemblies may be used with a variety of needle safety features, including a v-clip, simple clip, spring retraction, saf-t-intima type safety, among others.

Advantageously, one of more embodiments of disclosure provide a catheter assembly that is configured to place a longer indwelling, soft catheter using a traditional PIVC insertion method while ensuring proper hooding technique. One or more embodiments of the catheter assembly described herein are configured for use with robust needle safety mechanisms and provide higher flow rates than existing soft catheter alternatives.

One or more embodiments provide a catheter assembly with a hooding-only support sleeve advancement, meaning that the advancement assembly is configured to advance the catheter only a distance sufficient to hood the catheter over the distal tip of the introducer needle. In some embodiments the catheter can be advanced further by a user of the device manually. Advantageously, a hooding displacement force is applied via user input to cause the advancement assembly to advance the catheter by pivoting, distal rotation, proximal rotation, a squeeze, button, a slide mechanism, or a push button with spring element or other suitable means of advancement. Thus, in some embodiments, the catheter assembly comprises a means for activating the advancement assembly to move the catheter distal tip to a hooded position selected from the group consisting of a distal rotation activation feature, a proximal rotation activation feature, a squeeze mechanism, an activation button, a slide mechanism, and a push button with a spring element.

In some embodiments, a distal rotation activation feature operates so that the advancement assembly can be operated by rotating a component in a distal direction. In some embodiments, a proximal rotation activation feature operates so that the advancement assembly can be operated by rotating a component in a proximal direction. In some embodiment, a squeeze mechanism operates so that the advancement assembly can be operated by a device user applying squeezing force to advance the catheter. In some embodiments, an activation button operates so that the advancement assembly can be operated by pressing a button to activate the advancement mechanism and move the catheter. In some embodiments, a slide mechanism operates so that the advancement assembly can be operated by sliding a component that advances the catheter. In some embodiments, a push button with a spring element operates so that the advancement assembly can be operated by pressing push button combined with a spring element that bias the catheter in a distal direction.

In some embodiments, the catheter assembly may further comprise a guidewire for improved placement success. In some embodiments, the advancement assembly applies direct force to advancement feature coupled to the catheter distal tip support element. Alternatively, the advancement assembly applies force to a tip shield, which makes contact with the advancement feature coupled to the catheter distal tip support element.

In some embodiments, the distal tip of the catheter comprises fenestrations and/or a reinforced catheter tip, which provides a stiffer material at the distal tip of the catheter. In one or more embodiments, an antimicrobial additive is included in the catheter material. In one or more embodiments, the catheter distal tip support element is configured to cover the introducer needle tip to a provide needle safety to prevent accidental needle sticks. In other embodiments, the catheter assembly includes a needle safety mechanism.

Although the disclosure herein has been described with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

What is claimed is:

1. A catheter assembly configured for insertion into vasculature of a patient, the catheter assembly comprising:
a catheter including a catheter lumen and extending from a distal end of a catheter hub having a proximal end, the catheter, the catheter comprising:
a catheter stepped surface disposed within the catheter lumen and having a distal surface with a first inner diameter, a proximal surface with a second inner diameter greater than the first inner diameter, and a shoulder disposed between the distal surface and the proximal surface; and
an elongate tubular portion proximate the catheter stepped surface and having the second inner diameter of the proximal surface and a distal tip;
an introducer needle disposed within the catheter lumen, the introducer needle comprising a sidewall defining a proximal end, a distal tip, and a lumen extending through from the proximal end to the distal tip, the proximal end attached to a needle hub;
a catheter distal tip support element movably disposed within the catheter lumen and over the introducer needle, the catheter distal tip support element comprising a proximal end and a support material having a higher Shore durometer than a Shore durometer of a second material of the elongate tubular portion of the catheter, the catheter distal tip support element movable with respect to the catheter and the introducer needle and having a distal surface configured to contact the shoulder of the catheter and to advance the catheter in distal direction and prevent kinking or buckling of the second material of the elongate tubular portion as the catheter is advanced into the vasculature of the patient; and
an advancement assembly disposed between the needle hub and the distal tip of the introducer needle, the advancement assembly configured to advance the catheter distal tip support element a distance sufficient to advance the distal tip of the catheter to cover the distal tip of the introducer needle in a hooded position.

2. The catheter assembly according to claim 1, wherein the advancement assembly includes an advancement feature coupled to the catheter distal tip support element.

3. The catheter assembly according to claim 2, wherein the advancement feature is coupled adjacent to the proximal end of the catheter distal tip support element.

4. The catheter assembly according to claim 2, wherein the advancement feature extends transverse to the catheter distal support element.

5. The catheter assembly according to claim 4, wherein the advancement assembly further comprises a lever element engaged with the advancement feature.

6. The catheter assembly according to claim 5, wherein the lever element comprises a slot.

7. The catheter assembly of claim 6, wherein the advancement feature is engaged with the slot.

8. The catheter assembly according to claim 3, wherein lever element and the advancement feature are engaged with the catheter hub.

9. The catheter assembly according to claim 8, wherein the lever element and the advancement feature are engaged with the proximal end of the catheter hub.

10. The catheter assembly according to claim 7, wherein the lever element is pivotable about the advancement feature.

11. The catheter assembly according to claim 5, wherein pivoting the lever element advances the catheter distal tip support element a distance sufficient to advance the distal end of the catheter to cover the distal tip of the introducer needle in a hooded position.

12. The catheter assembly according to claim 11, wherein the lever element is pivotable in a distal direction.

13. The catheter assembly according to claim 11, wherein the lever element is pivotable in a proximal direction.

14. The catheter assembly according to claim 4, wherein the catheter distal tip support element comprises a sleeve.

15. The catheter assembly according to claim 14, wherein the shoulder, the proximal surface, and the distal surface are constructed of a first material having a Shore durometer equal to or greater than a Shore durometer of a second material of the elongate tubular portion.

16. The catheter assembly according to claim 15, wherein the sleeve comprises a sleeve material having a Shore durometer that is greater than the Shore durometer than the second material.

17. The catheter assembly according to claim 16, wherein the sleeve material comprises plastic.

18. The catheter assembly according to claim 16, wherein the sleeve material comprises metal.

19. The catheter assembly according to claim 4, wherein the advancement assembly comprises means for activating the advancement assembly.

20. The catheter assembly according to claim 4, wherein the advancement assembly includes means for activating the advancement assembly to move the distal tip of the catheter to a hooded position selected from the group consisting of an activation button, a sliding activation element, an activation lever, and a rotational activation mechanism.
